(19) United States
(12) Patent Application Publication
(10) Pub. No.: US 2023/0042520 A1
(43) Pub. Date: Feb. 9, 2023

(54) CANNULA SYSTEM FOR BODY FAT REMOVAL

(71) Applicant: OREL MEDICAL INC., Dollard-Des-Ormeaux (CA)

(72) Inventors: Moshe GABBAY, Dollard-des-Ormeaux (CA); David R. ROLSTON, Beaconsfield (CA)

(21) Appl. No.: 17/874,436

(22) Filed: Jul. 27, 2022

Related U.S. Application Data
(60) Provisional application No. 63/230,377, filed on Aug. 6, 2021.

(51) Int. Cl. A61M 1/00; A61N 5/06

(52) U.S. Cl. CPC A61M 1/89; A61N 5/0625; A61N 5/0601; A61M 2205/051; A61N 2005/0612; A61N 2005/0644; A61N 2005/0651

(57) ABSTRACT

The cannula can have a connector configured to be electrically connected to a source of electrical power; a tip comprising: a printed circuit board (PCB); a first LED operatively connected and attached to the PCB and configured to emit light in a first wavelength range; and a shell configured to allow the light in the first wavelength range to pass from the first LED outside of the shell; and a middle section located between the connector and the tip, the middle section comprising: a hollow tube attached to the shell, the tube having apertures for collecting a liquid fat into the tube.

### TECHNICAL FIELD

The present disclosure relates to body fat liquefaction and extraction with adipocyte preservation. More specifically, it relates to a cannula system for body fat liquefaction and extraction with adipocyte preservation.

### BACKGROUND

Obesity is a global epidemic that is quickly rising. Currently over 80% of people in North America are either overweight or obese, with proportion of obesity rising yearly. Additionally, the body composition is changing rapidly as the population becomes more sedentary, with more rapid loss of muscle mass, with the average of 1% per year, and gain in fat mass. Therefore, people in the overweight category may be classified as obese based on their high fat percent body composition.

Fat serves an important role in energy storage, with its main storage site being the adipocytes. The number of adipocytes and distribution throughout the body varies by person, and is responsible for the distinct apple shape (for men) and pear shape (for women) that may occur in the setting of excessive fatness.

The number of fat cells in the body is generally stable, with fatness occurring as a consequence of the adipocytes filling up rather than through increase in the number of fat cells. With increasing fatness, the adipocytes eventually reach their storage capacity. Once this occurs, the excess fat then deposits in other organs, including the muscles, liver, heart, blood vessels and in the abdominal cavity.

Deposition of fat in the organs leads to their impaired function, therefore leading to several known conditions such as diabetes, metabolic syndrome, hypertension, fatty liver, elevated cholesterol and heart failure, all of which contribute to significant morbidity, reduced quality of life, and higher risk of premature death. There is also impact on body mechanics related to excess weight, with increased risk for osteoarthritis and joint pains, as well as sleep apnea.

Current interventions for management of obesity include a variety of diets that focus on restriction of calories, and a variety of exercises, medications that help curb appetite, elimination of fat through stool, or boosting up metabolism, as well as various forms of bariatric surgery, or other interventions that serve to reduce the size of the stomach.

The most direct way to eliminate fat is with the use of liposuction techniques, which involve the insertion of a cannula and extracting the fat with suction. All the current liposuction techniques require mechanical motion of the cannula in parallel to the skin surface, leading to extensive damage to the structures within, including the fat cells, collagen, the nerves and blood vessels. In order to minimize blood loss, the current standard is the use of the tumescent technique, which involves the infiltration of large volumes of fluid and anesthetic to expand the fat compartment, with epinephrine in the solution acting to constrict the blood vessels, and reduce damage to blood vessels.

Liposuction techniques may use various modalities for easier extraction, such as laser, radiofrequency, ultrasound, and motorized cannulas. The purpose of all these techniques used is to destroy or remove the fat cells (adipocytes), often through the combination of heat, mechanical destruction, and aspiration of the fat cells resulting in reduction in the volume of the fat cells in the region treated.

The permanent removal of the fat cells, and their replacement with scar tissue, leads to desirable cosmetic results with the body area that was targeted.

The safety of the procedure significantly increased with the addition of a tumescent technique, which helped reduce the amount of blood loss, allowing for potentially larger volumes of fat removal, and faster recovery. Several studies have also demonstrated improvement in metabolic profile, including cholesterol levels after procedure.

However due to the destructive nature of the procedure and creation of scar tissue, the procedure is generally not repeated.

Additionally, if a person regains weight, the weight will accumulate in other parts of the body, which may lead to a dysmorphic appearance. Most importantly, the removal of the fat cells results in the removal of storage capacity within adipocytes. Therefore, any gain in fatness may result in its deposition in the organs, leading to a deterioration in health.

All the current liposuction techniques are considered cosmetic, with the ideal candidates being normal weight individuals with an area of the body has a bothersome cosmetic bulge. The procedure is considered a form of body sculpting, and not for management of weight, or obesity. Current guidelines recommend that fat removal be limited to no more than 5-6 kilograms.

Due to the inherent risks associated with liposuction, additional modalities for body sculpting have been developed over the last decade, using non-invasive techniques to either release the fat or to destroy the fat by delivering energy through the skin, such as radiofrequency, extreme cold, laser, ultrasound and others. All these techniques are also indicated for cosmetic body sculpting, and not for weight loss.

For management of obesity, we still need to preserve our ability to store energy, as an increasing number of adipocytes leads to increased storage capacity of energy, and reduces the need to store fat in the organs. Therefore, a removal of fat from the adipocytes, without their destruction, would allow for increased storage capacity for fat. Once adipocytes empty out, this would lead to fat redistribution in the body, with removal of the fat from essential organs, and shuttling it to the empty adipocytes, leading to improvement in the health and function of the body and the organs, leading to reduction in the risk for obesity related morbidity and mortality.

### SUMMARY

It is an object of the present disclosure to provide an apparatus and a method for fat liquefaction and extraction. The apparatus described herein causes adipocyte liquefaction, thereby removing adipocyte contents (i.e., the lipids in adipocytes), while preserving the integrity of the fat cells (adipocytes). The apparatus as described herein extracts the fat in a safer manner than currently available techniques, which may lead to significant reduction in injury from procedure, no destruction of tissue, leading to a faster recovery and better outcomes for patients (i.e., faster recovery after the procedure, more significant fat removal, fat removal in places where fat removal is more effective for the patient's health, etc.). This procedure may be indicated for fat removal, with the objective of management of obesity, rather than for body sculpting. However, the apparatus as described herein may be used as an adjunct to body sculpting procedures, acting as a debulking procedure that removes large amount of fat, prior to the sculpting process taking place.

In accordance with one aspect, there is provided: a cannula for fat liquification and extraction, the cannula comprising: a connector configured to be electrically connected to a source of electrical power; a tip comprising: a printed circuit board (PCB); a first LED operatively connected and attached to the PCB and configured to emit light in a first wavelength range; and a shell configured to allow the light in the first wavelength range to pass from the first LED outside of the shell; and a middle section located between the connector and the tip, the middle section comprising: a hollow tube attached to the shell, the tube having apertures for collecting a liquid fat into the tube.

In accordance with another aspect, there is provided an apparatus for fat liquification and extraction comprising: a cannula comprising: a connector; a tip comprising: a first LED configured to emit light in a first wavelength range; and a shell configured to allow the light in the first wavelength range to pass from the first LED outside of the shell; a middle section located between the connector and the tip, the middle section comprising: a hollow tube having apertures for collecting a liquid fat into the tube, and a wire located inside the tube, the wire configured to conduct electrical power from the connector to the first LED; and a cannula receptacle comprising: an electrical socket unit configured to provide electrical power to the connector and a fat collection unit configured to receive fat from the middle section of the cannula via the connector.

In some embodiments, a combination of the electrical socket unit and the fat collection unit are configured to fixedly attach the cannula to the cannula receptacle.

In accordance with another aspect, there is provided a cannula receptacle for attaching and using a cannula, the cannula comprising: a tip having a light source for liquifying fat; a hollow middle section with apertures for receiving a liquified fat, and a connector located on another side of the hollow middle section from the tip, the connector having a first conducting ring and a second conducting ring on an external surface of the connector; the cannula receptacle comprising: an electrical socket unit configured to provide electrical power to the connector, and a fat collection unit configured to receive the liquified fat collected in the hollow middle section of the cannula from an opening in the connector.

In accordance with another aspect, there is provided a multiple-cannula assembly comprising the cannula receptacle and another cannula receptacle, wherein tubing of the fat collection unit of the cannula receptacle is connected to the tubing of the another cannula receptacle.

In accordance with another aspect, there is provided a method of liquifying and removing fat, the method comprising: applying a power to an LED to liquify the fat, the LED being located in a tip of a cannula, and without removing the cannula from the fat, collecting a liquified fat in a middle section of the cannula.

### BRIEF DESCRIPTION OF THE DRAWINGS

Further features and advantages of the present disclosure will become apparent from the following detailed description, taken in combination with the appended drawings, in which:

FIG. 1 depicts a perspective view of a cannula, in accordance with at least one embodiment of the present disclosure;

FIG. 2 depicts a perspective view of a portion of the cannula of FIG. 1;

FIG. 3 depicts a perspective view of a cross-section of the tip of the cannula of FIG. 1;

FIG. 4 depicts another perspective view of the tip of the cannula of FIG. 1;

FIG. 5 depicts a perspective view of a connector of the cannula of FIG. 1;

FIG. 6A depicts a front view of the cannula of FIG. 1 showing the lumen thereof;

FIG. 6B depicts a rear view of the cannula of FIG. 1 showing the lumen thereof;

FIG. 7A depicts a cross-sectional view of a cannula receptacle with the cannula of FIG. 1 plugged therein forming a cannula unit, in accordance with at least one embodiment of the present disclosure;

FIG. 7B depicts a perspective view of the cannula unit of FIG. 7A;

FIG. 8A depicts a cross-sectional view of a cannula receptacle and the cannula of FIG. 1 to be plugged therein, in accordance with at least one embodiment of the present disclosure;

FIG. 8B depicts a cross-sectional view of the cannula receptacle with the cannula plugged therein forming the cannula unit of FIG. 7A;

FIG. 9 depicts an electrical connection and mechanical attachment of the cannula to the apparatus, in accordance with at least one embodiment of the present disclosure;

FIG. 10 depicts a transparent view of a cannula receptacle, in accordance with at least one embodiment of the present disclosure;

FIG. 11 depicts a cross-sectional view of the cannula receptacle of FIG. 10; and

FIGS. 12-14 depict a multiple-cannula assembly 500, in accordance with at least one embodiment of the present disclosure.

It will be noted that throughout the appended drawings, like features are identified by like reference numerals.

### DETAILED DESCRIPTION

Various aspects of the present disclosure generally address one or more of the problems of body fat extraction.

Current methods to extract fat from the patient's body begin with an initial incision using a “trocar”, a scalpel or a punch biopsy device to create a hole to introduce the cannula. Currently fat extraction requires a multi-step process that is initiated with tumescent infiltration with one set of cannulas and a different set of cannulas are used for the fat extraction procedure. Such a two-step process increases the likelihood for tissue injury. Moreover, to extract the fat during the liposuction procedures, mechanical back-and-forth motion needs to be performed using the conventional cannulas, which contribute to strain for the surgeon and increased tissue trauma.

Furthermore, one popular method for liposuction uses a laser system with optical fiber. These systems typically have only a limited number of wavelengths available which cannot be modified, which makes this cannula system not flexible relative to the wavelength of operation. Such currently conventional cannulas need a laser box for operation, which is expensive and bulky, and the surgeon is limited in the wavelengths that can be used

Using a light emitting device within the cannula system itself that can be introduced into the body and used from inside a body has not been yet proposed and is herein proposed as described further below.

A cannula, apparatuses and methods as described herein may provide for a safe extraction of fat from adipocytes, without removal or destruction of these adipocytes from which fat is extracted. The apparatus helps to avoid tissue damage, adipocyte removal, and formation of scar tissue, which typically limit the amount of fat which can be removed in a typical liposuction, allowing for the procedure to be repeated many times in the patient's life, including at the same place in the body and contrarily to standard liposuction procedures. It therefore allows removing larger amounts of fat during the cannula's service time (operation). The procedure may be safer than current techniques, and may allow for larger amounts of fat removal per session than in a typical liposuction session.

By preserving the fat cells/adipocytes, fat redistribution may occur throughout the body, leading the body to naturally re-establish equal distribution and proportionality, leading to fat lost from more distant areas, leading to better overall appearance, with reduction of fat in the face, neck, and arms and, overall body fat reduction as a result of the fat-extraction session using the cannula system according to an embodiment and as described further below.

The cannula and the apparatuses described herein have light emitting diodes (LEDs), and the user may choose the wavelength(s) of operation by selecting a cannula having a LED or LEDs with the proper wavelength of operation to cause a release of fat from adipocytes exposed to that proper wavelength with the option for an adjustable output optical power. The cannula and the apparatuses permit also the subsequent suction of the liquid fat which was just released from the adipocyte under the action of the LED(s). The cannula and the apparatuses described herein is a fully integrated cannula that has the light source incorporated into it, and the infiltration of the fluid and the fat suction all occur during one single insertion of the cannula into the body.

The cannula and the apparatuses described herein are versatile with different LED wavelengths that may be used simultaneously in different parts of the body, to cause different biological effects, notably those related to adipocyte fat release.

### Cannula—TIP

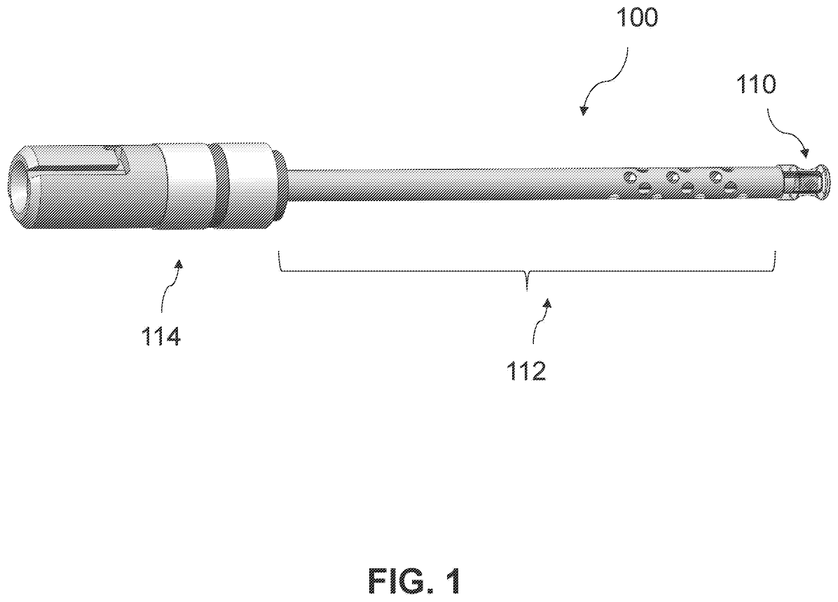

Referring now to the drawings, FIG. 1 depicts a cannula 100, in accordance with at least one embodiment of the present disclosure. Such cannula 100 may be used in a system for fat liquification and extraction, in accordance with at least one embodiment of the present disclosure.

The cannula comprises a tip 110, a middle section 112 and a connector 114.

### Cannula—LEDs

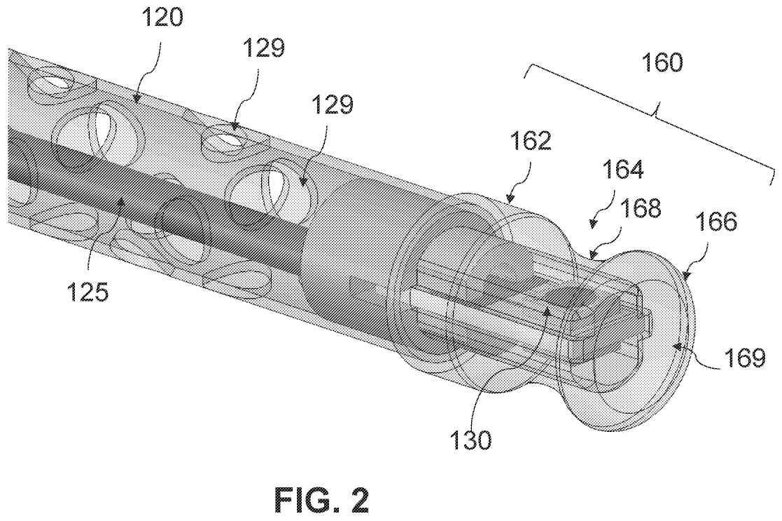

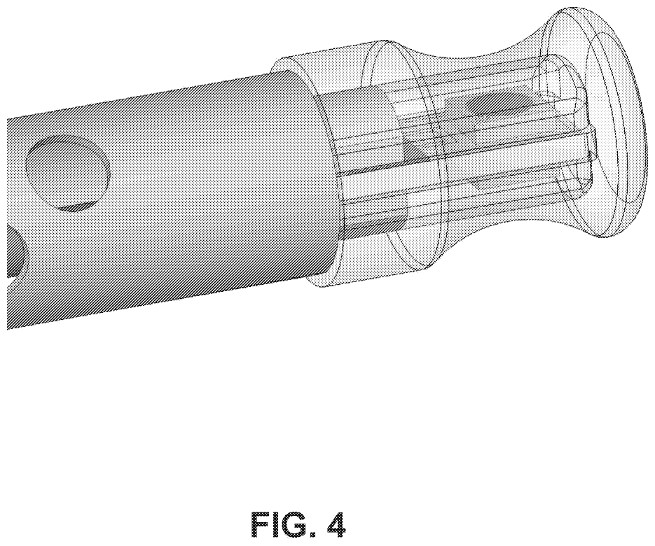

Referring also to FIGS. 2-4, the middle section 112 is located between the tip 110 and the connector 114 and comprises a tube 120 and a wire 125 located inside the tube 120.

The tip 110 comprises at least one light emitting diode (LED) 130, a printed circuit board (PCB) 140 for bringing the power to the LED(s) 130, and a shell 160. According to an embodiment of the present disclosure, the LED 130 allows for a wider diffusion of light in a greater volume, and also better wavelength selection (typically multiple bandwidth regions of the emitted light from various types of LED devices) all in the same cannula.

The tip 110 may also comprise a PCB holder 150. The implication is that the light source itself is introduced into the patient's body in proximity to the adipocytes to be acted upon for fat release. Using LEDs is advantageous because the wavelength can be selected when building the cannula 100 with a specific LED 130. Different cannulas 100 may also have a corresponding LED (or corresponding LEDs) having the same wavelength or different wavelengths, whereby a cannula 100 can be selected by the practitioner operating the system in view of the wavelength of its LED(s) 130 to be the most appropriate for a situation.

The cannula may have one or more LEDs 130 operatively connected and attached to the PCB 140 and configured to emit light. FIGS. 1-4 depict the cannula with two LEDs: a first LED 130a and a second LED 130b (referred to herein collectively as “LEDs 130” or “LED 130” when referred to any LED). Each one of the two LEDs 130 may be located on and attached to, for example, one of the two sides of the PCB 140. For example, one LED 130a may be located on one side of the PCB 140, and another LED 130b may be located on another side of the PCB 140 (opposed to the first one), as shown in FIGS. 2-4 and 6A.

The wavelengths of the light emitted by the LED 130 may be within the visible spectrum and/or the near infrared spectrum. For example, the LED 130 may emit wavelengths spanning from about 400 nanometers to about 2.5 microns.

According to an embodiment, the cannula may have more than two LEDs 130. For example, the more than one LED 130 may be located on each side of the PCB 140 if they are made long enough or large enough to accommodate more than one on a given side thereof. In some embodiment, the cannula 100 may have only one LED 130.

In some embodiments, the second LED 130b may be configured to emit light at the same wavelength or within the same wavelength range as the first LED 130a of the cannula 100 (if light is emitted at a rather large range about a given wavelength). In other embodiments, the second LED 130b may be configured to emit light at a wavelength different from the wavelength of the first LED 130a of the cannula 100.

The LED(s) 130 located in the tip 110 of the cannula 100 diffuse(s) the light in a circumferential manner thanks to their housing within a transparent light-diffusing shell which is described in more detail further below. The process of biophotomodulation causes release of fat from adipocytes (fat cells), while preserving the integrity of the cells. Biophotomodulation typically uses lower power light, typically less than 1000 milliwatts. The light emitted from LED 130 may also cause stimulatory effects on the cells, leading to improved metabolism. The light emitted from LED 130 also diffusing into surrounding structures, including the muscle, improves the metabolism, and also causes release of fat from the deeper structures, such as, for example, visceral fat. These light-induced physiological processes can take place more easily in view of the introduction of the light sources right inside the body at a location where the light-induced physiological processes should take place. Proper diffusion, light power control and also selection of appropriate wavelengths in view of the particular physiological processes to be induced by light further contribute in maximising the effect of the illumination.

A system using a cannula 100 with LEDs 130 rather than fiberoptic lasers may be cheaper in view of the low price, small size and ease of fabrication of LEDs in general and supporting PCB. A system using the cannula 100 with LED 130 may be disposable, in accordance with typical standards of medical or surgical procedures, which may provide and improve the sterility of procedure, and reduce the risk for cross-contamination. Each cannula 100 may only be used on a single patient, and instead of being strictly disposable, could be used on separate visits, following sterilization.

The power on the LEDs 130 is adjustable, depending on the body region use and depending on the outcome desired. For example, the power of LEDs 130 may be under 1000 mW per LED. The LED 130 may be operated with pulsed power or may be operated with varying power.

The process of adipocytes liquefaction and release as described herein may be used with a low-power LED, causing micropores to open in the fat cell wall, leading to the adipocyte emptying out of the fat into the interstitial space between the adipocytes, and preserving the integrity of the adipocytes.

In at least one embodiment, the light emitted from LED has a cone shape, which permits to increase the volume of fat cells responding to the light, with the cannula in place, to initiate the extraction of the fat, while minimizing the number of potential fat cells that would be aspirated. The fat extracted with the cannula 100 as described herein may be extracted from the interstitial space between the adipocytes where the liquified fat was released by illumination, without damage to blood vessels or the matrix, and no scar tissue formation.

### PCB and PCB Holder

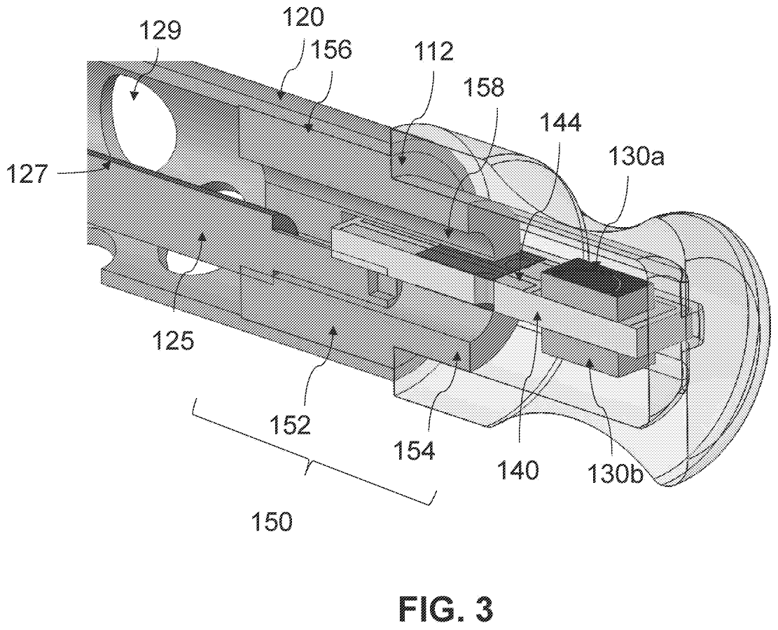

Referring to FIG. 3, the PCB 140 mechanically supports and electrically connects LEDs 130 using conductive tracks 144. The PCB 140 is electrically connected to the wire 125 acting as a stem and extending inside and along the length of the middle section 112 of the cannula 100.

The PCB holder 150 fits into (sits in) the distal portion of the tube 120. The PCB holder 150 has a tube holder portion 152 and a shell holder portion 154.

The tube holder portion 152 is adapted to be slidably mounted into cooperative engagement in conjoined, axially and radially fixed relation inside the extreme portion of the tube 120. The tube holder 152 portion is shaped such that an external surface 156 of the tube holder 152 is engaged in axial withdrawal-resisting relation inside the extreme portion of the tube 120.

The internal circumferential surface of the tube 120 is configured to be in an abutting relation with the generally circular cross-section holder body when the holder 150 is installed in the extreme portion of the tube 120. The external surface of the PCB holder 150 and the extreme portion of the tube 120 may be mutually secured by various arrangements, such as chemical bonding or glue.

Still referring to FIG. 3, the shell holder portion 154 of the PCB holder 150 has a diameter narrower than the diameter of the tube holder portion 152.

The tube holder abuts the internal surface of the tube 120.

The PCB 140 is attached to the PCB holder 150. For example, the PCB 140 may be located in a recess 158 in the PCB holder 150 and the PCB holder 150 may embrace two sides of the PCB 140. For example, the PCB 140 may be attached to the PCB holder 150 with a glue or soldered thereto.

### Cannula—Shell

Still referring to FIGS. 2-4, the shell 160 is configured to distribute and spread out the light emitted by the LEDs 130. In other words, the shell 160 is configured to allow the light in the first wavelength range to pass from the first LED 130a outside of the shell 160. In at least one embodiment, the shell 160 is transparent. While it may not be perfectly transparent, the level of transparency should be very high to ensure that light reaches the appropriate target in the body and avoid heating the shell 160 by light absorption.

According to an embodiment of the present disclosure, and as shown in FIG. 2, the shell 160 has a shell base 162, a radiator 164, and a shell dome or distal end 166. The shell base 162 is mounted into cooperative engagement in conjoined, fixed relation with the shell holder 154. The shell base 162 abuts the tube holder portion 152 and the tube 120 simultaneously while fixedly sitting on the shell holder portion 154 of the PCB holder 150.

As depicted in FIGS. 2-4, a radiator outside surface 168 of the radiator 164 of the shell 160 is concave. The outside surface of the radiator 164 or of the whole shell 160 may be textured. Such configuration of the shell 160 helps light scattering or diffusion. In some embodiments, light diffusion by the shell makes it a ball light with light emitted with a significant solid-angle range away from the tube 120.

The distal end 166 of the shell (which is also the distal end of the cannula), which houses the LED or LEDs, can be made to be blunted in a bullet-shape or other shape that minimizes tissue destruction and avoids penetration into vital organs. In some embodiments, the lateral surface 169 of the distal end 166 may be concave to help distribute and spread light.

The shell 160 may be made of an optically transparent material. The distal end 166, the radiator 164, and the shell base 162 may form one piece of the shell 160 and may be manufactured, for example, by molding.

The LEDs 130 are encased in the shell 160 that may be made of the polymer. The polymer is safe for using in human patients. The curvature of the shell 160 allows the diffusion of the light in a circumferential manner.

### Middle Section—Tube

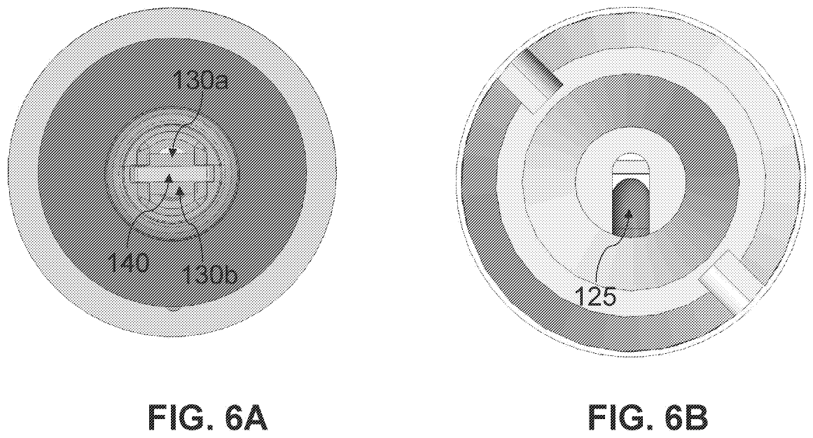

The wire 125 well shown in FIG. 2 and also in the rear view of FIG. 6B, may or may not be positioned coaxially with the tube 120. The wire 125 is configured to conduct electrical power from the connector 114 to the PCB 140.

The middle section 112 provides and electric connection between the connector and the tip. The tube 120 may serve as an electric ground. For example, the wire 125 may have a cladding 127 as shown in FIG. 3. For example, the wire 125 may bring a negative electrical connection to the LED 130, while the tube 120 itself may be grounded, thereby conducting electric power to the PCB 140 and LEDs 130 at the tip 110. Alternatively, the wire may in fact comprise two wires, cabled together or each in a separate cladding, for bringing the electrical power to the PCB 140 and therefore to the LED(s) 130. In some other embodiments, the electrical connection may be different.

According to an embodiment of the present disclosure, the tube 120 is attached to the shell 160 using the PCB holder 150.

The length of the cannula 100 may be, for example, between about 5 and about 20 cm, and preferably in the range of about 10 to about 15 cm. The length of the cannula 100, and therefore the length of the tube 120 may depend on the desired depth of penetration of the cannula 100 into the body.

Diameter of the tube 120 may be less than 3 mm. For example, the diameter of the tube 120 may be between 2 mm and 3 mm.

Optionally, additional modules, such as sensors (temperature, pressure or other body parameters) may be added inside the cannula 100. For example, such modules or sensors may be added inside the tube 120.

### Holes

The tube 120 is configured to receive liquid fat that was released from the adipocytes under the effect of light into the intracellular space around the tip 110, so that the tube 120 can receive said fat in its lumen and thereby extract the fat out of the body. The tube 120 has multiple apertures 129 which, during the operation of the cannula on a body, receives and collects the liquified fat into the tube 120. The apertures 129 are configured to allow the liquified fat to enter the tube 120 to be removed from the human body.

Having the apertures 129 on the tube 120, and having the apertures 129 in various locations along the length of the tube 120, may permit to increase the area from which the fat may be recuperated. Thus, the apertures 129 serve for both liquid infiltration and fat extraction.

The apertures 129 help to collect the liquified fat. The size of the apertures 129 is small enough to minimize the likelihood of suctioning fat cells.

### Connector

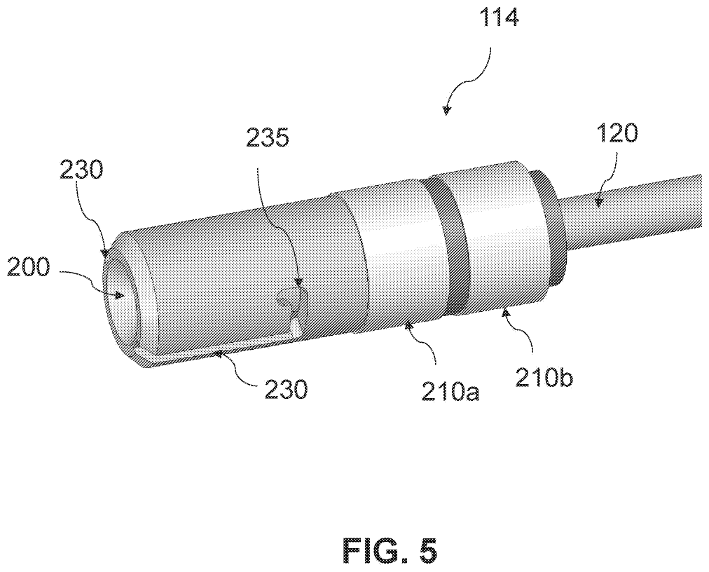

FIG. 5 depicts the connector 114 of the cannula 100, in accordance with at least one embodiment.

The connector 114 is hollow and has a cannula outlet 200 that allows the liquid that arrives from the tube 120 to the connector 114 to exit through the cannula outlet 200. For this, the connector 114 and the tube 120 are two communicating vessels. In some embodiments, the connector 114 is coaxial with the tube 120.

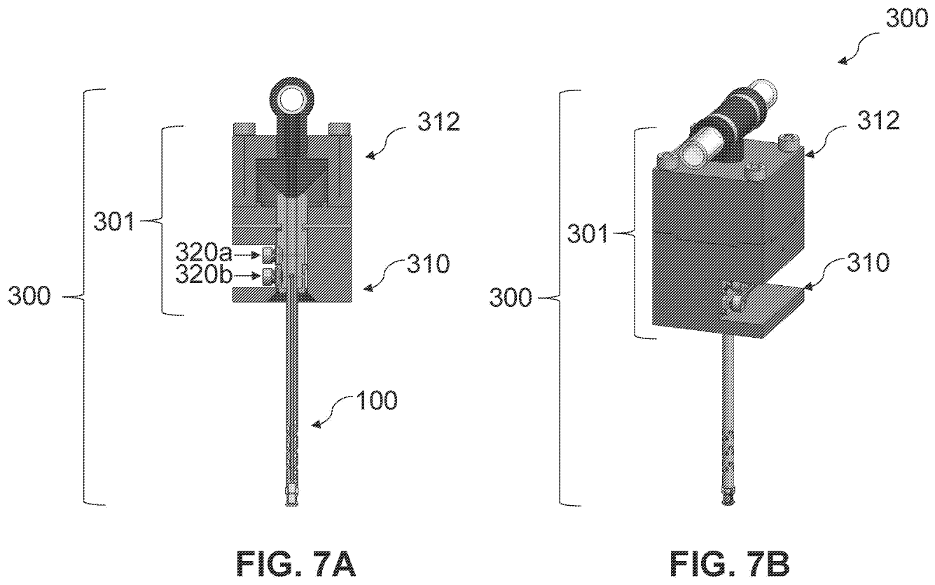

The connector 114 also has at least one side recess that is used for locking the cannula 100 in a cannula receptacle 300 (FIG. 7) upon introduction of the cannula 100 into the cannula receptacle 300, as described below. In FIG. 6, the connector 114 has two side recesses—one on each diametrically opposite side of the connector 114. The connector 114 (cannula outlet 200) is also configured to receive a cone-shaped portion of a cone of the cannula receptacle 300.

In FIG. 5, conducting rings 210a, 210b (also referred to herein as “rings” 210) are located on the surface of the connector 114 and provide electrical contacts for the electrical connection of the cannula 100, for the positive and negative electrical connections for powering the LEDs 130.

According to an exemplary embodiment of the present disclosure, the color of the rings 210 may be indicative of the wavelength (or the range of the wavelengths) of the LED 130 of the cannula 100 in order to make use of their visibility. For example, the user (such as a surgeon) may pick the cannula 100 with the wavelength he or she chooses based on said color. For example, each cannula 100 has its own wavelength or a range (combination) of wavelengths. In some embodiment, each cannula 100 may have a range of wavelengths. For example, a cannula 100 performing in a range of wavelengths may be implemented using two LEDs 130 on the PCB 140 inside the cannula tip 110 (one on top and one on the bottom).

The connector 114 of the cannula 100 connects to a fixed handle apparatus that provides the electricity to the LEDs 130. In other words, the connector 114 is configured to be electrically connected to a source of electrical power.

### Cannula—Operation

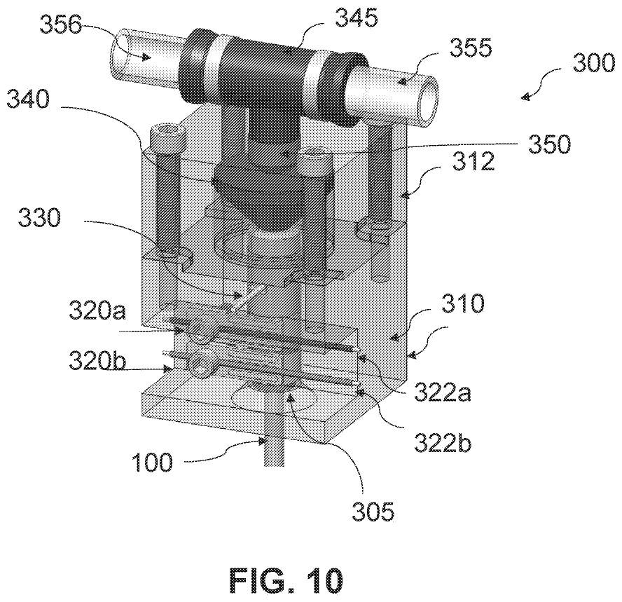

To operate, the cannula 100 needs to connect to a source of electrical power via the connector 114 and to a suction device, such as a pump, to facilitate suction of the fat. The tubing system connected to the pump may also serve to deliver a tumescent solution into the patient via the same cannula using tubing 356, shown for example in FIG. 10. An apparatus as described herein below may provide an electrical connection to the power source, facilitate suction of the fat from the cannula 100, and may help to manipulate the cannula 100 when recuperating the fat from the body.

Due to the materials and the LEDs 130 used in the cannula 100, the cannula 100 may be disposable.

### Cannula's Receptacle (Holding Apparatus)

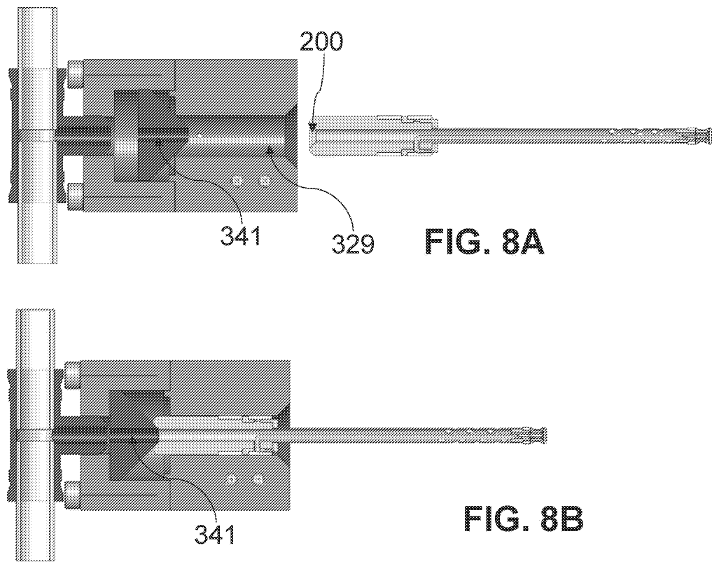

FIGS. 7A-8B depict a cannula receptacle 301 with a cannula 100 plugged therein, together forming a cannula unit 300, in accordance with at least one embodiment of the present disclosure.

The cannula receptacle 300 comprises an electrical socket unit 310 and a fat collection unit 312 which are attached to each other. For example, the electrical socket unit 310 and a fat collection unit 312 may be attached to each other using screws, as shown, although other ways to secure them together may be envisaged.

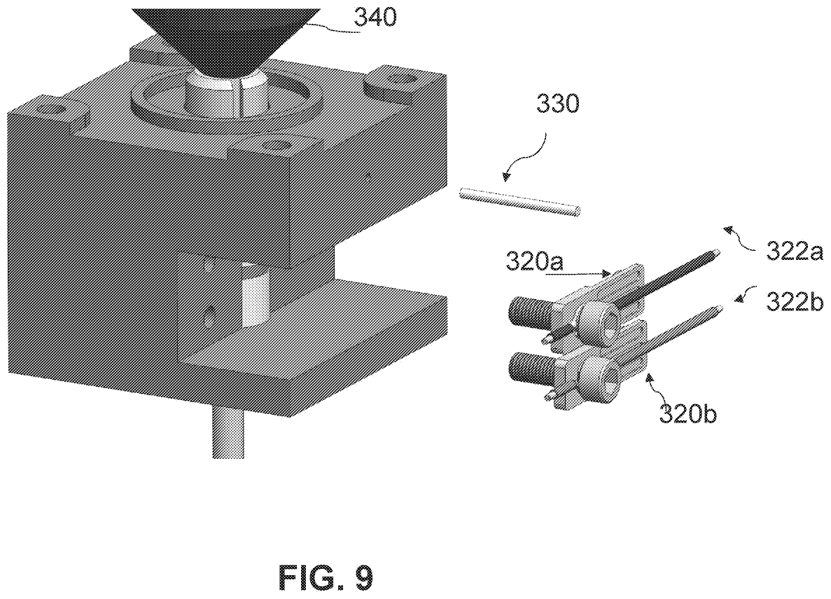

FIG. 9 depicts an electrical connection and mechanical attachment of the cannula 100 to the cannula unit 300, in accordance with at least one embodiment of the present disclosure.

The electrical socket unit 310 is configured to provide electrical power to the cannula 100. The electrical socket unit has two plates 320a, 320b attached to the body of the electrical socket unit 310 (for example, with screws). Each plate 320a, 320b is configured to touch one of two rings 210a, 210b of the connector 114 to electrically connect the ring 210 of the connector of the cannula to the corresponding external electrical wire 322a, 322b.

Referring to FIG. 9, connection 322a (blue) and connection 322b (red) may be electrical connections that, when the cannula 100 is installed in the cannula unit 300, permit to deliver electricity to the connection rings 210a, 210b of the cannula 100, in the end being respectively connected to the tube 120 acting as the ground and to the wire 125 for powering up the PCB 140 and the LEDs 130.

Referring also to FIG. 5, to maintain the cannula 100 in the cannula unit 300, an interlocking pin 330 locks into the side recess 230 of the connector of the cannula.

Referring now to FIGS. 8A, 8B, 9 and 10, to install the cannula 100 in the cannula unit 300, the cannula 100 is inserted linearly in translation into the bore 329 which is a lumen of an inner diameter corresponding to the outside diameter the base or connector 114 of the cannula 100 to fit it thereinto. When the cannula abuts the cone 340, the cannula 100 is rotated around its axis, to engage the pin 330 in the side recess 230 of the cannula 100. Referring also to FIG. 6, when the pin 330 reaches the pin abutment wall 235, it locks into the locking position with the locking recess 230. A click sound may be produced.

As depicted in FIG. 10, the electrical socket unit may have a cone-shaped entry 305 located at the place of entry of the cannula 100 into the cannula receptacle 300 to help guiding the cannula 100 into the cannula receptacle 300.

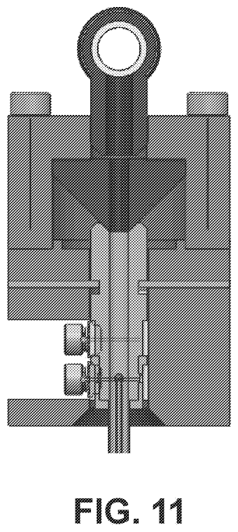

Referring now to FIGS. 10-11, the fat collection unit 312 comprises a cone 340, a T-junction 345, and a spring-rubber coned gasket 350.

When the cannula 100 is installed into the fat collection unit 312, the cone 340 located in the fat collection unit 312 mates to the cannula 100 and seals the connection with the spring-rubber coned gasket 350 and maintains mechanical tension to keep the cannula 100 well fitted in the cannula unit 300. For example, and without limitation, the cone 340 may be made of rubber.

Referring also to FIG. 5, the connector 114 has a cannula outlet 200 to allow the liquid that arrived from the tube 120 to the connector 114 to exit through the cannula outlet 200. The cone 340 contains a channel 341 (visible in FIGS. 8A-8B) which extends therethrough, from the apex of the cone 340, which collects fat from the cannula outlet 200 of the cannula's connector 114, to the upper-side base of the cone 340, which communicates with a bottom tube portion of the T-junction 345 which collects the fat having flowed through the channel 341 of the cone 230. To maintain proper vacuum at each of the distal ends of each cannula, proper care of vacuum flow must be designed into each area of the apparatus, otherwise risk of incomplete vacuum may occur.

Therefore, the connector 114 and the cone 340 are communicating and the fat from the connector 114 is delivered to the cone 340 and then, through the T-junction 345 to the liquid fat tubing 355.

FIG. 10 depicts the T-junction 345 and a tubing 355 for the evacuation of the liquid fat from the cannula 100, with appropriate tubing. As discussed above, pumping gear drives the flow of fat through the tubing 355 for aspiration of the fat from each plugged cannula. Given proper valve flow control in the tubing sections as well as the primary pumping system will also allow for the introduction of the tumescent solution into the patient using the same cannula and tubing 356.The tubing and pump system will accommodate for both the infiltration of the tumescent liquid and the extraction of liquified fat sequentially.

### Method of Operation of the Holding Apparatus

The cannula 100 and the cannula unit 300 may be used on a human or animal's body that has fat. For example, a local anesthetic may be first applied to all areas of cannula insertion to the live body. Then, the cannula 100 is introduced beyond the dermis into the fat layer. A tumescent solution may be used in the fat layer to expand the compartment, constrict the blood vessels, provide analgesia, and allow for easier extraction of the fat that is in the interstitial space.

The LED 130 is then activated at relatively low power (compared to prior art laser systems) for several minutes, delivered in a diffused cone pattern to expose larger volumes of fat tissue to the light. This leads to liquefaction of adipocytes in the area of light distribution.

After several minutes of powering the LED delivery, the same cannula 100 is activated with a suction mechanism, to extract the liquefied fat. The cannulas remain in place, and may be mechanically driven advanced in the same plane or retracted as well as individually rotated or micro-vibrated, in order to extract the fat from the surrounding tissue minimizing any possible blocking of the cannula's holes. Once the extraction has been completed, the cannulas are removed, and a dressing is applied.

For the cannula with different LEDs 130 in the visible and near infrared regions, power of various levels may be applied to the LEDs 130, and power of different levels may be applied to different LEDs of the same cannula 100.

When choosing the power level applied, the power should not cause destruction of the adipocytes, but rather preserve their integrity. Additional energy delivery devices may also be employed to achieve similar results, such as use of radiofrequency, ultrasound, or other energy delivery devices.

### Multiple-Canula Assembly/System

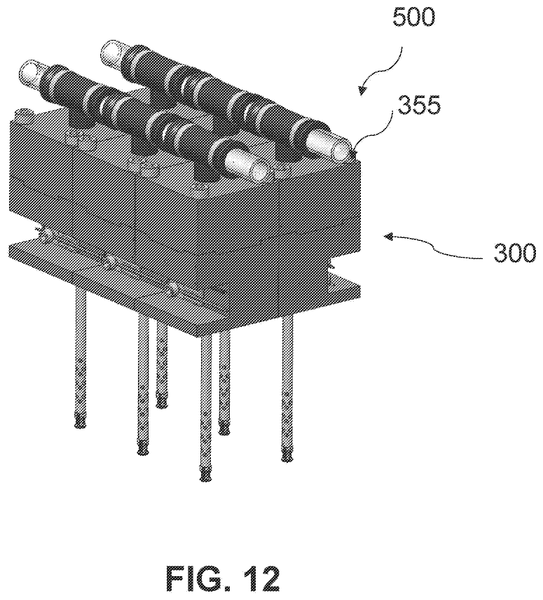

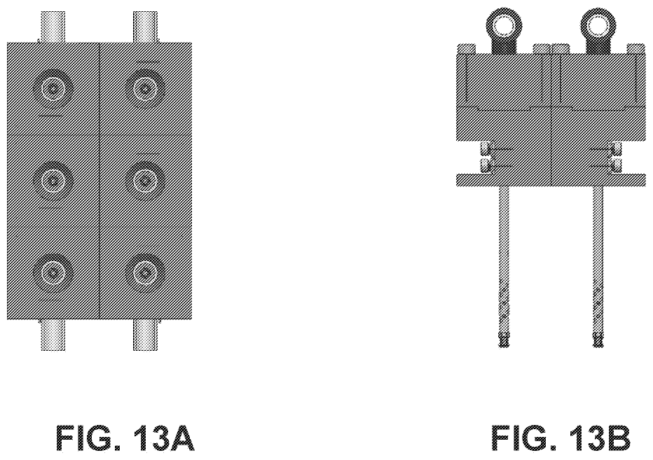

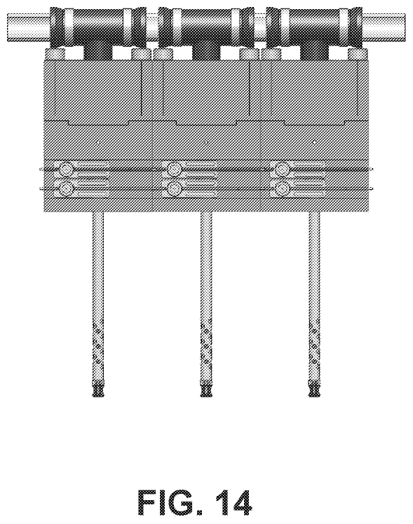

FIGS. 12-14 depict a multiple-cannula assembly 500, in accordance with at least one embodiment of the present disclosure. The multiple-cannula assembly 500 comprises two or more cannula units 300 stacked together. FIG. 500 depicts an example for six cannula units 300.

The cannula units 300 form apparatus rows, and the tubing portions 355 of two neighboring cannula units 300 in one apparatus row are connected to each other to form one tubing arrangement for receiving the liquid fat from the cannulas. The electrical wires that bring the electrical power to the electrical socket unit are also connected to each other for each two neighboring apparatuses in one apparatus row.

Depending on the number of cannula units 300, and set up of the multiple cannula system 500, connectors may be used between the tubes 355 to form a single tubing.

In some embodiments, the assembly may include spacers in between to modulate or adjust the distance between adjacent cannulas if needed. Alternatively, one or more apparatuses of the multiple-cannula assembly 500 may have a dummy cannula in the array. Having such a dummy cannula may be needed to maintain the proper suction in the tubing 355 while having no cannula somewhere, hence the need for the dummy cannula.

In some embodiments, one side of the tube may have a cap, others may be joined with T-junction. The tubing of the multiple-cannula assembly is connected to a pump.

Additional versatility may be ensured by connecting various cannulas together, and therefore allowing a larger surface area to be treated, and larger volume of the liquid fat to be extracted, in a shorter period of time. This may result in an improved comfort of the patient during and after the procedure.

When using the multiple cannula system, different wavelengths may be used with different cannulas, to have a synergistic effect, being able to treat the fat, cellulite, muscle, and collagen all at the same time. All these effects would lead to a combination of fat loss, increase in metabolism, reduction in visceral fat, and skin tightening, all occurring simultaneously.

In one holding apparatus, and/or or in one multiple-canula system, a variety of cannulas may be used, with various shapes, sizes, lengths, and number of ports, all of which serve to extract the fat.

There are different ways to perform suction, and the cannula, the holding apparatus and the multiple-canula system as described herein may use a variety of suction devices available, such as, for example, handheld suction devices or mechanical suction devices.

In some embodiments, flow sensors can be used in the tube 120 or in the tubing 355 to assess flow, heat sensors can be used to detect overheating of parts, especially those inserted in the body, to detect early if faulty parts should be removed, and resistance sensors may be used to detects abnormalities when they occur, for example.

### Other Advantages

The cannula system as described herein does not need any mechanical back-and-forth motion parallel to the skin's surface during the fat liquefaction procedures. The cannula system has an integrated LED system that liquefies the fat by a low power LED.

The cannula system as described herein is configured to liquify the fat and subsequently provide suction of that liquified fat, all through the same cannula. Multiple cannulas may be operating simultaneously, allowing for larger volumes of fat to be extracted over a shorter period of time.

There is no time limit for fat extraction, as the process may be continued until a predetermine safe amount of liquified fat is extracted from that specific region of the body. The cannula may reach progressively deeper areas of fat, without extracting the cannula from the body.

There is no downtime for the patient after the procedure. The patient's recovery may be rapid (faster than typical liposuction techniques).

For example, several body parts may be treated either sequentially or simultaneously. The procedure may be repeated every few weeks until desired amount of fat loss has been attained.

The apparatuses and methods as described herein may be used for liquifying and removing fat in vivo and in vitro in people, animals, as well as any other application where liquifying the fat may be needed. For example, food industry.

1. A cannula for fat liquification and extraction, the cannula comprising:
a connector configured to be electrically connected to a source of electrical power;
a tip comprising:
a printed circuit board (PCB);
a first LED operatively connected and attached to the PCB and configured to emit light in a first wavelength range; and
a shell configured to allow the light in the first wavelength range to pass from the first LED outside of the shell; and
a middle section located between the connector and the tip, the middle section comprising:
a hollow tube attached to the shell, the tube having apertures for collecting a liquid fat into the tube.

2. The cannula of claim 1, wherein at least one portion of the shell is concave.

3. The cannula of claim 1, wherein the shell comprises a base section, a dome section, and a radiation section located between the base section and the dome section, and the radiation section is concave.

4. The cannula of claim 1, wherein a dome section of the shell is concave.

5. The cannula of claim 1, wherein the shell is translucent.

6. The cannula of claim 1, wherein the PCB is a double-sided PCB, the first LED being attached to one side of the double-sided PCB, and the tip further comprises another LED operatively connected to and attached to a second side of the double-sided PCB.

7. The cannula of claim 6, wherein the second LED is configured to emit light within the same wavelength range as the first LED.

8. The cannula of claim 7, further comprising a wire located inside the tube, the wire configured to conduct electrical power from the connector to the PCB.

9. The cannula of claim 8, wherein the cannula further comprises a PCB holder, and the hollow tube and the wire are attached to the shell with a PCB holder.

10. A cannula receptacle for attaching and using the cannula of claim 1, the cannula receptacle comprising:
an electrical socket unit configured to provide electrical power to the connector, and
a fat collection unit configured to receive a liquified fat collected in the hollow middle section of the cannula from an opening in a connector of the cannula.
